(19) United States
(12) Patent Application Publication
(10) Pub. No.: US 2006/0035918 A1
(43) Pub. Date: Feb. 16, 2006

(54) Use of tacrolimus (fk506) derivatives combined with beta2-agonists for the treatment of asthma

(75) Inventors: Yoshitaka Hirayama, Osaka (JP); Yoshihiko Morishita, Osaka (JP)

Correspondence Address: LEYDIG VOIT & MAYER, LTD, 700 THIRTEENTH ST. NW, SUITE 300, WASHINGTON, DC, 20005-3960, US

(73) Assignee: Fujisawa Pharmaceutical Co., Ltd., Osaka-shi (JP)

(21) Appl. No.: 10/528,420

(22) PCT Filed: Nov. 4, 2003
(86) PCT No.: PCT/JP03/14077

§ 371 Date: Mar. 18, 2005

(30) Foreign Application Priority Data
Nov. 8, 2002 (AU) 2002952559

(51) Int. Cl. A61K 31/4745; A61K 31/16; A61K 31/137

(52) U.S. Cl. 514/291; 514/630; 514/649

(57) ABSTRACT

A method for treating or prevent acute or chronic asthma is disclosed, comprising administering effect amounts of an FK506 derivative and a β2-agonist to a human being or an animal. A composition comprising an FK506 derivative and a β2-agonist as a combined preparation is also disclosed

### TECHNICAL FIELD

This invention relates to a new combination use of FK506 derivatives and β2-agonist, which is useful in a medical field.

### BACKGROUND ART

Despite recent advances in the awareness of asthma and the introduction of powerful and effective anti-asthma drugs, asthma remains a poorly understood and frequently poorly treated disease. There have been recent advances in the treatment of the disease which result from the recognition that asthma is a chronic inflammatory disease. Therapy is now aimed at both controlling the symptoms and reducing the inflammation. The symptoms may be controlled by β2-agonists such as terbutaline, salbutamol, formoterol and salmeterol. Prophylactic therapy is typically provided by steroids such as beclomethasone dipropionate, fluticasone propionate, mometasone furoate and budesonide.

In spite of modern maintenance treatment too many asthmatic patients are undertreated for a number of reasons with a negative impact on their quality of life. Too complicated therapy with different medications and devices may lead to misunderstanding and communication problems between patient and doctor. Poor compliance is a common phenomenon. Improved patient education may partly counteract this, but does not completely solve the problem. A new and simpler approach to asthma treatment could thus be of tremendous help for many patients suffering from respiratory disease, particularly asthma. The combination of budesonide and formoterol in the same device as suggested in PCT applications WO 93/11773 and WO 98/15280 (both to Astra AB of Sweden) offers a favorable pathway to improve today's asthma management with an excellent safety profile.

FK506 derivatives, such as tacrolimus and its related compounds, are known to have preventing or treating reversible obstructive airways disease, such as asthma (U.S. Pat. No. 5,519,049). And an aerosol formulation comprising FK506 derivatives are also known by U.S. Pat. No. 6,361,760.

### DISCLOSURE OF INVENTION

This invention relates to a new use of FK506 derivatives and β2-agonist for manufacturing a medicament for simultaneous, separate or sequential use for treating or preventing acute or chronic asthma.

And further, this invention also relates to a method for treating or preventing acute or chronic asthma , by administering an effective amount of FK506 derivatives and β2-agonist, simultaneously, separately or sequentially, to a human being or an animal.

A further object of this invention is to provide a composition comprising FK506 derivatives and β2-agonist as a combined preparation for treating and preventing acute or chronic asthma.

And this invention also relates to the followings.

A use of FK506 derivatives for manufacturing a medicament for treating or preventing acute or chronic asthma with β2-agonist, simultaneously, separately or sequentially.

A composition comprising FK506 derivatives for treating or preventing acute or chronic asthma with β2-agonist, simultaneously, separately or sequentially.

A use of β2-agonistfor manufacturing a medicament for treating or preventing acute or chronic asthma with FK506 derivatives, simultaneously, separately or sequentially.

A composition comprising β2-agonist for treating or preventing acute or chronic asthma with FK506 derivatives, simultaneously, separately or sequentially.

In the present invention, the “β2-agonist” should not be limited and be considered to mean any compounds which can stimulate β2receptor. Preferably, long-acting β2-agonists (such as, salmeterol, formoterol, etc) and short-acting β2-agonists (such as albuterol, bitolterol, fenoterol, isoetharine, metaproterenol, pirbuterol, terbutaline, salbutamol, etc) can be exemplified. More preferable one is long-acting β2-agonists, such as, salmeterol, or formoterol.

The “FK506 derivatives” means tricyclic compounds shown in EP-0184162, WO89/05303, WO93/05058, WO96/31514, and so on, the disclosure of which is incorporated herein by reference. It is well known that those tricyclic compounds have strong immunosuppressive activity.

As a particular example of the tricyclic compounds, the tricyclic compound of the following formula (I) can be exemplified. 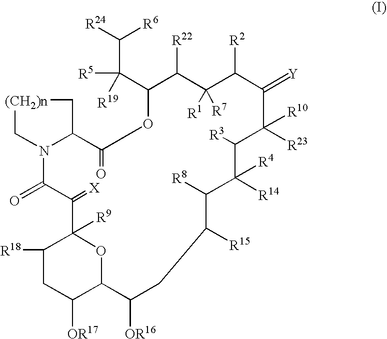
(wherein each of adjacent pairs of R¹ and R², R³ and R⁴, and R⁵ and R⁶ independently

(a) is two adjacent hydrogen atoms, but R² may also be an alkyl group or

(b) may form another bond formed between the carbon atoms to which they are attached;

R⁷ is a hydrogen atom, a hydroxy group, a protected hydroxy group, or an alkoxy group, or an oxo group together with R¹;

R⁸ and R⁹ are independently a hydrogen atom or a hydroxy group;

R¹⁰ is a hydrogen atom, an alkyl group, an alkyl group substituted by one or more hydroxy groups, an alkenyl group, an alkenyl group substituted by one or more hydroxy groups, or an alkyl group substituted by an oxo group;

X is an oxo group, (a hydrogen atom and a hydroxy group), (a hydrogen atom and a hydrogen atom), or a group represented by the formula —CH₂O—;

Y is an oxo group, (a hydrogen atom and a hydroxy group),
(a hydrogen atom and a hydrogen atom), or a group represented by the formula N—NR¹¹R¹² or N—OR¹³;

R¹¹ and R¹² are independently a hydrogen atom, an alkyl group, an aryl group or a tosyl group;

R¹³, R¹⁴, R¹⁵, R¹⁶, R¹⁷, R¹⁸, R¹⁹, R²² and R²³ are independently a hydrogen atom or an alkyl group;

R²⁴ is an optionally substituted ring system which may contain one or more heteroatoms;

n is an integer of 1 or 2; and

in addition to the above definitions, Y, R¹⁰ and R²³, together with the carbon atoms to which they are attached, may represent a saturated or unsaturated 5- or 6-membered nitrogen, sulfur and/or oxygen containing heterocyclic ring optionally substituted by one or more groups selected from the group consisting of an alkyl, a hydroxy, an alkoxy, a benzyl, a group of the formula —CH₂Se(C₆H₅), and an alkyl substituted by one or more hydroxy groups.

The definitions used in the above general formula (I) and the specific and preferred examples thereof are now explained and set forth in detail.

The term “lower” means, unless otherwise indicated, a group having 1 to 6 carbon atoms.

Preferable examples of the “alkyl groups” and an alkyl moiety of the “alkoxy group” include a straight or branched chain aliphatic hydrocarbon residue, for example, a lower alkyl group such as methyl, ethyl, propyl, isopropyl, butyl, isobutyl, pentyl, neopentyl and hexyl.

Preferable examples of the “alkenyl groups” include a straight or branched chain aliphatic hydrocarbon residue having one double-bond, for example, a lower alkenyl group such as vinyl, propenyl (e.g., allyl group), butenyl, methylpropenyl, pentenyl and hexenyl.

Preferable examples of the “aryl groups” include phenyl, tolyl, xylyl, cumenyl, mesityl and naphthyl.

Preferable protective groups in the “protected hydroxy groups” and the “protected amino” are 1-(lower alkylthio)-(lower)alkyl group such as a lower alkylthiomethyl group (e.g., methylthiomethyl, ethylthiomethyl, propylthiomethyl, isopropylthiomethyl, butylthiomethyl, isobutylthiomethyl, hexylthiomethyl, etc.), more preferably C₁-C₄ alkylthiomethyl group, most preferably methylthiomethyl group;

trisubstituted silyl group such as a tri(lower)alkylsilyl (e.g., trimethylsilyl, triethylsilyl, tributylsilyl, tert-butyldimethylsilyl, tri-tert-butylsilyl, etc.) or lower alkyl-diarylsilyl (e.g., methyldiphenylsilyl, ethyldiphenylsilyl, propyldiphenylsilyl, tert-butyldiphenyl-silyl, etc.), more preferably tri(C₁-C₄)alkylsilyl group and C₁-C₄ alkyldiphenylsilyl group, most preferably tert-butyldimethylsilyl group and tert-butyldiphenylsilyl group; and an acyl group such as an aliphatic, aromatic acyl group or an aliphatic acyl group substituted by an aromatic group, which are derived from a carboxylic acid, sulfonic acid or carbamic acid.

Examples of the aliphatic acyl groups include a lower alkanoyl group optionally having one or more suitable substituents such ascarboxy, e.g., formyl, acetyl, propionyl, butyryl, isobutyryl, valeryl, isovaleryl, pivaloyl, hexanoyl, carboxyacetyl, carboxypropionyl, carboxybutyryl, carboxyhexanoyl, etc.; a cyclo(lower)alkoxy(lower)alkanoyl group optionally having one or more suitable substituents such as lower alkyl, e.g., cyclopropyloxyacetyl, cyclobutyloxypropionyl, cycloheptyloxybutyryl, menthyloxyacetyl, menthyloxypropionyl, menthyloxybutyryl, menthyloxypentanoyl, menthyloxyhexanoyl, etc.; a camphorsulfonyl group; or a lower alkylcarbamoyl group having one or more suitable substituents such as carboxy or protected carboxy, for example, carboxy(lower)alkylcarbamoyl group (e.g., carboxymethylcarbamoyl, carboxyethylcarbamoyl, carboxypropylcarbamoyl, carboxybutylcarbamoyl, carboxypentylcarbamoyl, carboxyhexylcarbamoyl, etc.), tri-(lower)alkylsilyl(lower)alkoxycarbonyl(lower)alkylcarbamo yl group (e.g., trimethylsilylmethoxycarbonylethylcarbamoyl, trimethylsilylethoxycarbonylpropylcarbamoyl, triethylsilylethoxycarbonylpropylcarbamoyl, tert-butyldimethylsilylethoxycarbonylpropylcarbamoyl, tri-methylsilylpropoxycarbonylbutylcarbamoyl, etc.) and so on.

Examples of the aromatic acyl groups include an aroyl group optionally having one or more suitable substituents such as nitro, e.g., benzoyl, toluoyl, xyloyl, naphthoyl, nitrobenzoyl, dinitrobenzoyl, nitronaphthoyl, etc.; and

an arenesulfonyl group optionally having one or more suitable substituents such as halogen, e.g., benzenesulfonyl, toluenesulfonyl, xylenesulfonyl, naphthalenesulfonyl, fluorobenzenesulfonyl, chlorobenzenesulfonyl, bromobenzenesulfonyl, iodobenzenesulfonyl, etc.

Examples of the aliphatic acyl groups substituted by an aromatic group include ar(lower)alkanoyl group optionally having one or more suitable substituents such as lower alkoxy or trihalo(lower)alkyl, e.g., phenylacetyl, phenylpropionyl, phenylbutyryl, 2-trifluoromethyl-2-methoxy-2-phenylacetyl, 2-ethyl-2-trifluoromethyl-2-phenylacetyl, 2-trifluoromethyl-2-propoxy-2-phenylacetyl, etc.

More preferable acyl groups among the aforesaid acyl groups are C₁-C₄ alkanoyl group optionally having carboxy, cyclo(C₅-C₆)alkoxy(C₁-C₄)alkanoyl group having two (C₁-C₄)alkyls at the cycloalkyl moiety, camphorsulfonyl group, carboxy-(C₁-C₄)alkylcarbamoyl group, tri(C₁-C₄)alkylsilyl(C₁-C₄)alkoxycarbonyl(C₁-C₄)-alkylcarbamoyl group, benzoyl group optionally having one or two nitro groups, benzenesulfonyl group having halogen, or phenyl(C₁-C₄)alkanoyl group having C₁-C₄ alkoxy and trihalo(C₁-C₄)alkyl group. Among these, the most preferable ones are acetyl, carboxypropionyl, menthyloxyacetyl, camphorsulfonyl, benzoyl, nitrobenzoyl, dinitrobenzoyl, iodobenzenesulfonyl and 2-trifluoromethyl-2-methoxy-2-phenylacetyl.

Preferable examples of the “5- or 6-membered nitrogen, sulfur and/or oxygen containing heterocyclic ring” include a pyrrolyl group and a tetrahydrofuryl group.

R²⁴ is an optionally substituted ring system which may contain one or more heteroatoms, Preferable R²⁴ maybe cyclo(C₅₋₇)alkyl group optionally having suitable substituents, and the following ones can be exemplified.

(a) a 3,4-di-oxo-cyclohexyl group;

(b) a 3-R²⁰-4-R²¹-cyclohexyl group,
in which R²⁰ is hydroxy, an alkoxy group, an oxo group, or a —OCH₂OCH₂CH₂₀OCH₃ group, and
   R²¹ is hydroxy, —OCN, analkoxy group, a heteroaryloxy which may be substituted by suitable substituents, 1- or 2-tetrazolyl, a —OCH₂OCH₂CH₂OCH₃ group, a protected hydroxy group, chloro, bromo, iodo, aminooxalyloxy, an azido group, p-tolyloxythiocarbonyloxy, or R²⁵R²⁶CHCOO—,
   in which R²⁵ is optionally protected hydroxy or protected amino, and
      R²⁶ is hydrogen or methyl, or
R²⁰ and R²¹ together form an oxygen atom in an epoxide ring; or

(c) cyclopentyl group substituted by methoxymethyl, optionally protected hydroxymethyl, acyloxymethyl
(in which the acyl moiety optionally contains either a dimethylamino group which may be quaternized, or a carboxy group which may be esterified), one or more amino and/or hydroxy groups which may be protected, or aminooxalyloxymethyl. A preferred example is a 2-formyl-cyclopentyl group.

“A heteroaryl which may be substituted by suitable substituents” moiety of the “heteroaryloxy which may be substituted by suitable substituents” may be the ones exemplified for R¹ of the compound of the formula of EP-A-532,088, with preference given to 1-hydroxyethylindol-5-yl, the disclosure of which is incorporated herein by reference.

The tricyclic compounds (I) and its pharmaceutically acceptable salt for use in accordance with this invention are well known to have excellent immunosuppressive activity, antimicrobial activity and other pharmacological activities and, as such, be of value for the treatment or prevention of rejection reactions by transplantation of organs or tissues, graft-vs-host diseases, autoimmune diseases, and infectious diseases [EP-A-0184162, EP-A-0323042, EP-A-423714, EP-A-427680, EP-A-465426, EP-A-480623, EP-A-532088, EP-A-532089, EP-A-569337, EP-A-626385, WO89/05303, WO93/05058, WO96/31514, WO91/13889, WO91/19495, WO93/04680, WO93/5059, etc.], the disclosures of which are incorporated herein by reference.

Particularly, the compounds which are designated as FR900506 (=FK506), FR900520 (ascomycin), FR900523, and FR900525 are products produced by microorganisms of the genus Streptomyces, such as Streptomyces tsukubaensis No. 9993 [deposited with National Institute of Bioscience and Human Technology Agency of Industrial Science and Technology (formerly Fermentation Research Institute Agency of Industrial Science and Technology ), at 1-3, Higashi 1-chome, Tsukuba-shi, Ibaraki, Japan, date of deposit Oct. 5, 1984, accession number FERM BP-927] or Streptomyces hygroscopicus subsp. yakushimaensis No.7238 [deposited with National Institute of Bioscience and Human Technology Agency of Industrial Science and Technology (formerly Fermentation Research Institute Agency of Industrial Science and Technology ), at 1-3, Higashi 1-chome, Tsukuba-shi, Ibaraki, Japan, date of deposit Jan. 12, 1985, accession number FERM BP-928] [EP-A-0184162]. The FK506 (general name: tacrolimus) of the following chemical formula, in particular, is a representative compound. 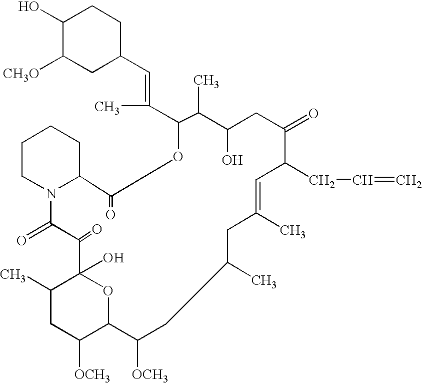
Chemical Name:
17-allyl-1, 14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0^{4,9}]octacos-18-ene-2,3,10,16-tetraone

The preferred examples of the tricyclic compounds (I) are the ones, wherein each of adjacent pairs of R³ and R⁴ or R⁵ and R⁶ independently form another bond formed between the carbon atoms to which they are attached;

each of R⁸ and R²³ is independently a hydrogen atom;

R⁹ is a hydroxy group;

R¹⁰ is a methyl group, an ethyl group, a propyl group or an allyl group;

X is (a hydrogen atom and a hydrogen atom) or an oxo group;

Y is an oxo group;

each of R¹⁴, R¹⁵, R¹⁶, R¹⁷, R¹⁸, R¹⁹, and R²² is a methyl group;

R²⁴ is a 3-R²⁰-4-R²¹-cyclohexyl group,
in which R²⁰ is hydroxy, an alkoxy group, an oxo group, or a —OCH₂OCH₂CH₂OCH₃ group, and
R²¹ ishydroxy, —OCN, analkoxy group, a heteroaryloxy which may be substituted by suitable substituents, 1- or 2-tetrazolyl, a —OCH₂OCH₂CH₂OCH₃ group, a protected hydroxy group, chloro, bromo, iodo, aminooxalyloxy, an azido group, p-tolyloxythiocarbonyloxy, or R²⁵R²⁶CHCOO—,
   in which R²⁵ is optionally protected hydroxy or protected amino, and
      R²⁶ is hydrogen or methyl, or
R²⁰ and R²¹ together form an oxygen atom in an epoxide ring; and

n is an integer of 1 or 2.

The most preferable tricyclic compounds (I) is, in addition to FK506, ascomycin derivatives such as halogenated-ascomycin (e.g., 33-epi-chloro-33-desoxyascomycin), which is disclosed in EP 427,680, example 66a.

The tricyclic compounds(I) may be in a form of its salt, which includes conventional non-toxic and pharmaceutically acceptable salt such as the salt with inorganic or organic bases, specifically, analkali metal salt such as sodium salt and potassium salt, an alkali earth metal salt such as calcium salt and magnesium salt, an ammonium salt and an amine salt such as triethylamine salt and N-benzyl-N-methylamine salt.

With respect to the tricyclic compounds of the present invention, it is to be understood that there may be conformers and one or more stereoisomers such as optical and geometrical isomers due to asymmetric carbon atom(s) or double bond(s), and such conformers and isomers are also included within the scope of the present invention. And further, the tricyclic compounds can be in the form of a solvate, which is included within the scope of the present invention. The solvate preferably include a hydrate and an ethanolate.

While the effective dosage of FK506 derivatives depends on the type of the said FK506 derivatives, the patient's age, type of disease, severity of illness, and other factors, a daily dose thereof is about from 0.001 to 10000 μg, preferably from 0.01 to 1000 μg, and more preferably, from 0.1 to 500 μg for therapeutic purposes. The average unit dose may be generally about 0.1 μg, 0.5 μg, 1 μg, 5 μg, 10 μg, 50 μg, 100 μg, 250 μg, or 500 μg.

A suitable unit dose of β2-agonist is in the range of from 0.1 μg to 500 μg, preferably from 0.5 μg to 250 μg, and more preferably between 1 μg to 100 μg. The daily dose of β2-agonist, such as formoterol (as fumarate dihydrate), including maintenance therapy, should be in the range of from 0.1 μg to 1000 μg, preferably from 0.5 μg to 500 μg, and more preferably from 1 μg to 200 μg.

The particular dose regimen will depend on the patient (age, sex, weight etc.) and the severity of the disease (mild, moderate, severe asthma etc.).

Preferably the mixture comprises one or more pharmaceutically acceptable additives, diluents or carriers, more preferably in an amount of from 50 μg to 4000 μg in each dose, most preferably in an amount of from 100 μg to 2000 μg and most preferably from 100 μg to 1000 μg. Examples of suitable additives, diluents or carriers include lactose, dextran, mannitol or glucose. Preferably lactose is used, and more preferably as the monohydrate.

One or more of the ingredients of the mixture may be in the form of dry powder, more preferably a small particle dry powder, most preferably an agglomerated small particle dry powder. Alternatively one or more of the active ingredients are in the form of an ordered mixture with diluent, additive or carrier. The ingredients used in the invention can be obtained in these preferred forms using methods known to those skilled in the art. The particle size of the active ingredients is preferably less than 10 μm.

Administration may be by inhalation, orally or intranasally. The ingredients of the system are preferably adapted to be administered from a dry powder inhaler, a pressurized metered dose inhaler, or a nebulizer. When the ingredients of the system are adapted to be administered from a pressurized inhaler, they are preferably in a small particle form. They are dissolved, or, preferably, suspended in a liquid propellant mixture. The propellants which can be used include chlorofluorocarbons, hydrocarbons or liquefied hydrofluoroalkane. Especially preferred propellants are HFA-134a (tetrafluoroethane) and HFA-227, each of which may be used alone or in combination. They are optionally used in combination with one or more other propellants and/or one or more surfactants and/or one or more other excipients, for example ethanol, a lubricant, an antioxidant and/or a stabilizing agent.

When the ingredients of the system of the invention are adapted to be administered via a nebulizer they may be in the form of a nebulized aqueous suspension or solution, with or without suitable pH or tonicity adjustment, either as a unit dose or multidose formulation.

If advisable, β2-agonist can be mixed with the FK506 derivatives prior to its use. So, the composition comprising the said β2-agonist of the present invention may further comprise the FK506 derivatives. And optionally, it comprises further additional active ingredients.

The following Examples are given for the purpose of illustrating the present invention in detail.

### EXAMPLE 1

Assay for inhibitory activity against respiratory resistance, antigen-induced airway inflammation and airway hyper-responsiveness.

Method

(1) Preparation of Antigen-Sensitized Guinea Pigs

Ovalbumin (OA)-sensitized guinea pigs were prepared in a similar manner to that of Am. J. Respir. Crit. Care Med. 160 (2): 663-671 (1999).

(2) Assay for Respiratory Resistance, Antigen-Induced Airway Inflammation and Airway Hyper-Responsiveness

Drugs can be given to animals placed in a plastic chamber by puffing aerosol of the drugs. Then, aerosolized OA solution is introduced in the chamber. Antigen-induced immediate increase in airway resistance can be monitored in a similar manner to that of Eur J Pharmacol (1996) April 11;300 (3):215-9.

On the next day, the airway responsiveness to acetylcholine can be determined in mice in a similar manner to that of J. Exp. Med. (1998) 188: 157-167.

After sacrifice, bronchoalveolar lavage (BAL) can be conducted and the cells in the BAL fluid can be collected and differentially counted.

### EXAMPLE 2

Method

(1) Immunization

Hartley guinea pigs weighing approximately 300 g were injected with saline solution of egg albumin (EA, 5 mg/mL) intraperitoneally and subcutaneously. The same procedure was repeated 7 days after the first immunization.

(2) Antigen Challenge

Animals were challenged with EA 7 or 8 days after the second immunization. A conscious animal was placed in a double chambered box, which consists of a nasal chamber and a body chamber. EA solution (1% in saline) nebulized into the nasal chamber using an ultra sonic nebulizer for 3 min. Saline instead of EA solution was used for the negative control group. All animals received 10 mg/kg of pyriramine maleate intraperitoneally 35 min before the antigen challenge.

(3) Drug Inhalation

FK506 aerosol (2 puffs) identified below, its placebo (4 puffs), Serevent® (salmeterol xinafoate) inhalation aerosol (2 puffs) identified below, or FK506+Serevent® (each 2 puffs) was given once, 30 min before the antigen challenge. Animals were placed in the double chambered box, puffed aerosol, and kept for 2 min.
FK506 aerosol (0.1%): FK506 (5 mg) in a mixture of HFA-134a and Miglyol 812, which was prepared in a similar manner to Example 1 of U.S. Pat. No. 6,361,760.
Serevent®: salmeterol xinafoate (36.25 μg/1 puff) in a mixture of 2 chlorofluorocarbon propellants (trichlorofluoromethane and dichlorodifluoromethane) with soya lecithin.
   (4) Antigen-Induced Immediate Response

Immediate bronchoconstriction was assessed by measurement of enhanced pause (Penh) as described in Reference 1). After measurement of basal value of Penh for 5 min, animals were challenged with EA or saline, and then measured Penh for 10 min.

(5) Antigen-Induced Late Response

Accumulation of eosinophils in airways 24 hr after the antigen challenge was assessed. After the animals were sacrificed, bronchoalveolar lavage (BAL) was repeated three times through a tracheal cannula, and the BAL fluid (BALF) was collected. The BALF cells were obtained by centrifugation, suspended with saline, the total cell number counted, and the cell suspension was smeared on a slide glass. The cells were stained and differentially counted intoneutrophils, eosinophils, macrophages, lymphocytes and others under a microscope. To obtain the absolute number of each cell type in the BALF, the percentages of each cell type were multiplied by the total numbers of cells recovered from the BALF.

### RESULTS AND CONCLUSION

The results were summarized in Table 1 and Table 2.

Antigen inhalation caused an immediate bronchoconstriction and a late response (airway inflammation). The combinatory use of FK506 aerosol and Serevent® inhalation aerosol suppressed both the immediate response and the late response.

TABLE 1
Effect of FK506 aerosol and Serevent ® inhalation
aerosol on antigen-induced immediate bronchoconstriction.

| | | Antigen | | % increase in Penh |
|---|---|---|---|---|
| Drug | puffs | challenge | n | above base line |
| — | — | — | 5 | 33 ± 17* |
| Placebo | 4 | + | 7 | 507 ± 127 |
| FK506 aerosol | 2 | + | 8 | 630 ± 171 |
| Serevent ® | 2 | + | 8 | 61 ± 38* |
| FK506 aerosol + | 2 + 2 | + | 8 | 66 ± 7* |
| Serevent ® | | | | |

*P < 0.05 vs Placebo group (t test)
Mean ± S.E.

TABLE 2
Effect of FK506 aerosol and Serevent ® inhalation
aerosol on antigen-induced airway inflammation.

| | | | | Number of |
|---|---|---|---|---|
| | | Antigen | | eosinophils (×105 |
| Drug | puffs | challenge | n | cells/animal) |
| — | — | — | 5 | 3.77 ± 1.06** |
| Placebo | 4 | + | 7 | 71.99 ± 16.82 |
| FK506 aerosol | 2 | + | 8 | 16.36 ± 2.81** |
| Serevent ® | 2 | + | 8 | 44.44 ± 6.53 |
| FK506 aerosol + | 2 + 2 | + | 8 | 7.92 ± 1.98 |
| Serevent ® | | | | **, #, && |

**P < 0.01 vs Placebo group (t test)
Mean ± S.E.
#: P < 0.05 vs FK506 aerosol group (t test)
&&: P < 0.01 vs Serevent ® group (t test)

Therefore, the combinatory therapy with FK506 and Serevent® has a great advantage over the therapy with FK506 alone or Serevent® alone in asthma.

### REFERENCE

1) E. HAMELMANN, J. SCHWARZE, K. TAKEDA, A. OSHIBA, G. L. LARSEN, C. G. IRVIN, and E. W. GELFAND
Am. J. Respir. Crit. Care Med., Volume 156, Number 3, September 1997, 766-775 Noninvasive Measurement of Airway Responsiveness in Allergic Mice Using Barometric Plethysmography.

### INDUSTRIAL APPLICABILITY

From the above invention, it is confirmed the combination use of FK506 derivatives and β2-agonist shows a remarkable and/or synergistic prevention of asthmatic attack upon antigen exposure, relief of on-going bronchospasm, reduction of airway hyper-responsiveness and reduction of airway inflammation, which leads to better control of the condition of asthma patients. The combination use is also useful for decreasing side effects of FK506 derivatives and/or β2-agonist by providing a better control and thus by decreasing the total amount of each drug.

From another aspect, the present invention also provides the following inventions.

i) An article of manufacture, comprising packaging material and FK506 derivatives and β2-agonist contained within said packaging material, wherein said FK506 derivatives and β2-agonist is therapeutically effective for treating and preventing acute or chronic asthma, and

wherein said packaging material comprises a label or a written material which indicates that FK506 derivatives and β2-agonist can be used for treating and preventing acute or chronic asthma.

ii) An article of manufacture, comprising packaging material and FK506 derivatives and β2-agonist contained within said packaging material, wherein said FK506 derivatives and β2-agonist is therapeutically effective for treating and preventing acute or chronic asthma, and

wherein said packaging material comprises a label or a written material which indicates that said FK50 6 derivatives and β2-agonist can be used for treating and preventing acute or chronic asthma.

The patents, patent applications and publications cited herein are incorporated by reference.

1. (canceled)

2. A method for treating or preventing acute or chronic asthma, by administering an effective amount of an FK506 derivative and an effective amount of a β2-agonist to a human being or an animal.

3. A composition comprising an FK506 derivative and a β2-agonist as a combined preparation.

4. The method of claim 2, wherein the FK506 derivative is tacrolimus or its hydrate.

5. The method of claim 2, wherein the β2-agonist is salmeterol or formoterol.

6-9. (canceled)

10. The method of claim 2, comprising simultaneously administrating the FK506 derivative and the β2-agonist to the human being or the animal.

11. The method of claim 2, comprising separately administrating the FK506 derivative and the β2-agonist to the human being or the animal.

12. The method of claim 2, comprising sequentially administrating the FK506 derivative and the β2-agonist to the human being or the animal.

13. The composition of claim 3, wherein the FK506 derivative is tacrolimus or its hydrate.

14. The composition of claim 3, wherein the β2-agonist is salmeterol or formoterol.

15. The composition of claim 13, wherein the β2-agonist is salmeterol or formoterol.

16. The method of claim 4, wherein the β2-agonist is salmeterol or formoterol.
